(19) United States
(12) Patent Application Publication
(10) Pub. No.: US 2008/0228210 A1
(43) Pub. Date: Sep. 18, 2008

(54) SYSTEM, METHOD AND DEVICE FOR CORNEAL MARKING

(76) Inventor: Andrew Peter Davis, Bellevue, WA (US)

Correspondence Address: BLACK LOWE & GRAHAM, PLLC, 701 FIFTH AVENUE, SUITE 4800, SEATTLE, WA, 98104, US

(21) Appl. No.: 12/047,261

(22) Filed: Mar. 12, 2008

Related U.S. Application Data
(60) Provisional application No. 60/906,461, filed on Mar. 12, 2007.

(51) Int. Cl. A61F 9/007

(52) U.S. Cl. 606/166; 606/1

(57) ABSTRACT

A system, method and device for marking the eye pre-operatively. Specifically, an embodiment of the invention is a corneal marking device assembly mounted onto a trial frame apparatus that can be adjusted by using the adjustment features on the trial frame apparatus. The corneal marking device assembly provides the stabilized structure through which the surgeon can observe the eye, measure characteristic features of the eye and mark the corneal surface with a corneal marking device housed in the corneal marking device assembly.

### PRIORITY CLAIM

This application claims priority from earlier filed U.S. Provisional Patent Application Ser. No. 60/906,461 filed Mar. 12, 2007. The foregoing application is hereby incorporated by reference in its entirety as if fully set forth herein.

### FIELD OF THE INVENTION

This invention relates generally to an ocular marking system and marking device and, more specifically, a corneal marking system and device to mark the corneal surface of the eye prior to a surgical procedure to correct the ocular condition of astigmatism.

### BACKGROUND OF THE INVENTION

Astigmatism, an optical defect which blurs vision, is usually caused by an irregular curvature of the cornea. In a perfectly shaped eye, the cornea is spherical, and bends all incoming light in the same way, producing a sharply focused image on the back of your eye (retina). In individuals with astigmatism, the cornea is assymetrically curved, similar to the surface of a football, with one axis of the ball curved more steeply than the opposite axis of the ball. This causes the light rays to be bent differently, which causes the image to be blurred.

The blurred vision from the astigmatism can be measured and is designated as the astigmatic refractive error. The astigmatic refractive error is measured in terms of its power and axis. The astigmatic power is measured in diopters, and the axis is measured in degrees representing the direction on a 360 degree scale. The axis reflects the direction of the steepest or flattest meridian of the cornea. The axis of astigmatism is unique to each individual.

The goal of treating astigmatism is to address the uneven curvature that causes blurred vision. Astigmatism may be corrected with eyeglasses, contact lenses, or surgery. Surgical correction of astigmatism can include laser (such as Lasik or Prk) as well as LRI (Limbal Relaxing Incisions, which are deep incisions in the peripheral cornea that flatten the steep meridian) and astigmatic intraocular lenses (at the time of cataract surgery).

A cataract is a condition where the normally clear lens of the eye becomes progressively opaque. This opacification typically occurs over an extended period of time, and the amount of light that passes through the lens decreases with increasing degrees of opacity. As the ability of the cataract lens to transmit light decreases the ability of the eye to perceive images also decreases. Blindness ultimately can result. Since there are no known methods for eliminating the opacity of a cataract lens, it generally is necessary to surgically remove the opaque lens to permit the unobstructed passage of light through the pupil to the retina.

In cataract surgery, the cloudy natural lens is removed from the eye. The focusing power of the natural lens can be restored by replacing it with a permanent artificial lens or intraocular lens (IOL) implant. These lenses are placed in the eye and thus closely simulate the optics of the natural lens which they are replacing. During cataract surgery, the astigmatic error can be corrected by either performing Limbal Relaxing Incisions in the peripheral cornea to flatten the steeper meridian, or by inserting a specialized IOL which can correct the astigmatism (a toric IOL).

Regardless of whether astigmatism is corrected with an LRI or with a toric intraocular lens, it is essential to align the surgical intervention with the exact astigmatic axis. Prior to the surgery it is necessary to mark the astigmatic axis onto the patient's cornea as accurately as possible.

A variety of methods and instruments are currently used to mark the cornea prior to surgery. Most involve a small handheld instrument (e.g., U.S. Pat. No. 6,217,596) that is pressed against the cornea prior to surgery, marking reference horizontal or vertical axis. After the eye surgery has started, and the patient is lying down, the reference marks are used to mark the cornea a second time at the direction of the astigmatic axis. This methodology has several shortcomings and introduces several significant sources of error.

Holding the marking instrument with a free hand to mark the cornea naturally leads to small but significant unwanted movement as the instrument approaches the cornea. There are unpreventable movements in both the horizontal and vertical direction and also rotational movements which contribute to error.

Another source of error is the inherent difficulty in trying to estimate the vertical or horizontal plane by free hand. Some markers have a small level gauge on the handle to assist in keeping the marker horizontal or a small weight and a marker that orients like a plumb bob to keep the marker vertical. Yet even with these methods the patient often tilts their head when the lids are held open during the marking procedure, introducing additional error. Therefore, it is very challenging for the surgeon to simultaneously keep the marker centered on the cornea, hold the marker level, keep the marker stable, and make sure that the patient's head is vertical, all while holding the patient's lid up.

Another source of error is introduced by the use of a fixation light during the procedure. A surgeon will often use a fixation light to help the patient keep their direction of gaze at one point, allowing the surgeon to use the mirror image of the fixation light on the cornea as a center point for the marking instrument. The use of such a fixation light usually requires an assistant to help hold the patient's lids open, introducing further error (as well as the need for an assistant). Additionally, it is difficult to hold the fixation light steady while holding the marker steady, while also aiming for such a tiny reference point on the cornea.

Moreover, error is introduced when the surgeon uses the alignment reference marks to then mark the astigmatic axis. While the alignment is done while the patient is sitting upright, the marking is done while the patient is lying down. The eye undergoes movement within the socket comprising translation and rotation (“cyclotorsion”) as the patient is moved from the upright measuring position to the prone surgery position. Multiple techniques known in the art to accommodate this movement include those disclosed in U.S. Pat. No. 4,476,862 and U.S. Pat. No. 4,705,035. If the eye movement is not taken into consideration when the patient lies down the direction of the axis of astigmatism will not be properly accounted for. Mathematically, missing the axis of astigmatism to be treated by 10 to 15 degrees can lead to a treatment under-correction of 50% or more.

Finally, the change in orientation encountered with the patient lying down with surgeon now approaching the patient from the side or from the forehead can be inherently confusing when attempting to mark the axis which was measured with the patient sitting. Difficulty with centration and rotation compound the multiple errors already introduced.

The prior art of free hand corneal marking is unreliable, non-precise and non-reproducible. There exists then, an unmet need in the art to minimize errors resulting from the methods and tools employed for marking the eye in a systematic way.

### SUMMARY OF THE INVENTION

The present invention addresses the systematic errors introduced by marking the cornea using a hand-held marking device. The invention reduces errors that can result from, for example, a patient's movement, a surgeon's movement, errors of estimation of horizontal and vertical axes and/or errors of estimating centration.

The corneal marking system and method of the present invention comprise a means for illuminating the eye to observe the corneal light reflex produced by the illuminating means; a means for aligning the corneal marking device with an unobstructed corneal surface according to the corneal light reflex using the mounting apparatus adjustment controls; and a means for marking the eye.

In one embodiment of the present invention the means for aligning the corneal marking device is a trial frame apparatus. In an alternative embodiment, the means for marking the eye is a corneal marking component that receives ink to be delivered to a corneal surface of the eye when in a close proximity with the eye.

One embodiment of the present invention includes a corneal marking device assembly for corrective eye surgery having a base member configured to mount on a mounting apparatus; a guide member defining a marking path along which a corneal marking device can pass towards a patient's eye, the guide member having first and second ends. The first end receives the corneal marking device and the second end is attachable to the base member in relative proximity to the patient's eye; at least one guide slot extending along a length of the guide member, where the guide slot orients the corneal marking device in fixed rotational position along a marking path; and a compression mechanism in physical association with the corneal marking device that provides resistance against the corneal marking device as it moves along the marking path.

Another embodiment of the present invention includes a corneal device for marking an eye prior to a corrective surgical procedure having a housing with two ends, the first end having a marking component for marking the corneal surface of the eye and a crosshairs reference to align the corneal marking device and the second end configured to provide a light source defining a marking path associated with the reflex of light reflected by the eye; and at least one rotational positioning member configured to fix the corneal marking device along the marking path.

The present invention is further directed to a method for marking a corneal surface of an eye of a patient, the method including the steps of orienting a mounting apparatus having position adjustment controls relative to the patient's eye; illuminating the eye with a light source; observing the corneal light reflex produced by the light source; aligning a corneal marking device associated with the mounting apparatus with an unobstructed corneal surface according to the corneal light reflect using the mounting apparatus adjustment controls; and marking the unobstructed corneal surface of the eye.

As will be readily appreciated from the foregoing summary, the present invention provides an improved corneal marking system, device and method to mark the corneal surface of the eye prior to a surgical procedure to correct the ocular condition of astigmatism and thus improve the outcome of astigmatic refractive surgeries.

### BRIEF DESCRIPTION OF THE DRAWINGS

Preferred and alternative examples of the present invention are described in detail below with reference to the following drawings:

FIG. 1 is a perspective view of the preferred embodiment corneal marking device assembly of the present invention;

FIG. 2 is a perspective view of the one embodiment of the corneal marking device of the present invention from one end configured to receive the light source;

FIG. 3 is a perspective view of the one embodiment of the corneal marking device from an opposite end from that shown in FIG. 2, showing the marking structures and crosshairs of the cornea marking end;

FIG. 4A is a perspective view of the corneal marking device assembly and the corneal marking device as it would appear inside the guide tube;

FIG. 4B is an elevation view of the corneal marking device assembly and the corneal marking device including the marking and crosshairs components at the corneal marking end as it would appear inside the guide tube, as viewed from and through the base end;

FIG. 5 is a perspective view of the corneal marking device assembly held in place by a mounting apparatus, in this example a trial frame apparatus known to be standard equipment in most ophthalmic offices, further showing the corneal marking device, and the light source;

FIG. 6 is a perspective view of a light source, in this example a handheld transillumination device as is known to be standard equipment in most ophthalmic offices;

FIG. 7 is a perspective view of an alternative embodiment for the corneal marking device where the light source, in this example the shaft of a transillumination device is fitted with a bumper that engages the housing of the corneal marking device inside the guide member;

FIG. 8 is a perspective view of an alternative embodiment for the corneal marking device where the corneal marking device is fitted to the light source, in this example a transillumination device, shown here with a rotational positioning member and marking component;

FIG. 9 is a perspective view of an alternative embodiment for the corneal marking device where an illumination LED is fitted onto the rotational positioning member of the marking component of the corneal marking device; and

FIG. 10 is a flow diagram of the methodology of the present invention.

### DETAILED DESCRIPTION OF THE PREFERRED EMBODIMENT

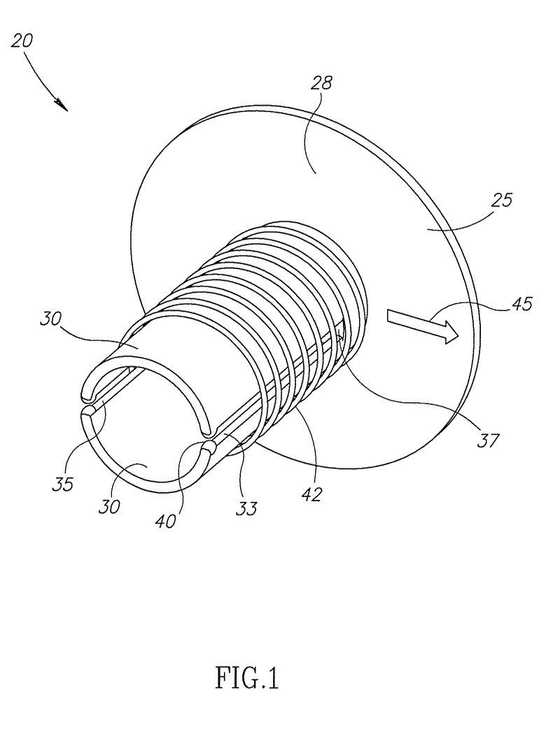

A description of the preferred embodiments of the present invention will now be presented with reference to FIGS. 1-10.

In a preferred embodiment, the corneal marking device assembly of the present invention is fitted to a mounting assembly, for example, a standard trial lens assembly of a trial frame apparatus to be used while the patient is sitting upright. Because the corneal marking device assembly is mounted onto the mounting frame, preferably a trial frame apparatus, the corneal marking device assembly is adjustable using the adjustment features on the trial frame apparatus. The corneal marking device assembly provides a stabilized apparatus structure through which a surgeon can observe the eye, measure characteristic features of the eye and mark the corneal surface of the eye pre-operatively. Using an illumination device, which may be any type of external light source, for example a flashlight or penlight, or a transillumination device more typically available in the industry, or a light source integrated with the invention, the surgeon can focus light into the patient's eye through a guide member of the corneal marking device assembly and thereafter adjust and align the assembly according to the resultant corneal light reflex of the observed eye. The surgeon, while in the same position, can contact the eye with the corneal marking device by pushing the transillumination device against the corneal marking device through the guide member on the corneal marking device assembly and along the marking path. As used herein, “marking path” is used to describe the corridor that centers on the corneal light reflected by the light source. The transillumination device maintains the patient's direction of gaze coaxial to the guide member while at the same time pushing the housing of the corneal marking device forward in the marking path. As the marking component comes in contact with the corneal surface, marks are made on the surface. The marks on the corneal surface are preferably applied by painting the marker component with dyes, which are transferred upon contact with the corneal surface. The preferred means for achieving this systematic method are detailed herein.

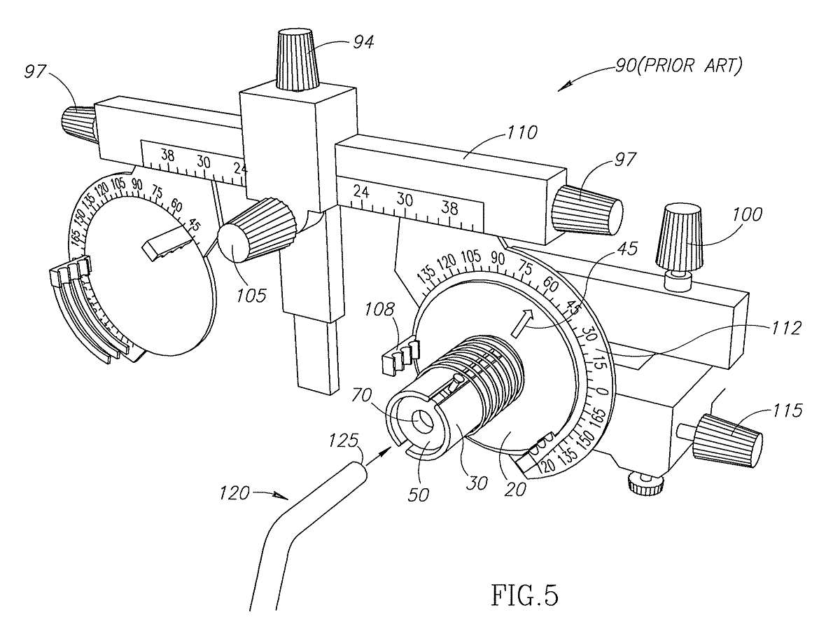

FIG. 1 shows a preferred embodiment of a corneal marking device assembly 20 comprising a circular base 25 that is of a diameter, thickness and edge contour comparable to a standard trial lens, as is known in the art. In this nonlimiting embodiment, the corneal marking device assembly 20 can be held in place by a lens holder mounting assembly (FIG. 5, 108) on a trial frame apparatus (FIG. 5, 90). It is further contemplated that the means by which the corneal marking device assembly 20 of the present invention is stabilized and secured into place is a mounting assembly other than a trial frame apparatus, for example, any mounting assembly configured to observe, center, align, and adjust the corneal marking device assembly 20. A trial frame or an alternative mounting assembly provides the means for aligning the corneal marking device (FIG. 1 50) associated with the corneal light reflex of the eye along a marking path or, in other words, the corridor that centers on the corneal light reflected by the light source.

At the base's center is a guide member 30 that is perpendicular to and attached to the base 25, on face 28 of the circular base 25. In this example, the guide member 30 has two linear slots 33, 35 beginning at the front of the guide member 30. The linear slots run substantially down the length of the guide member 30 to point 37. A small stricture 40 may be located at the open end of the linear slots 33, 35. In this nonlimiting example, a compression spring 42 surrounds the guide member 30 and rests against the base 25. A linear directional mark 45 is located on the base 25, extending radially from the center of the base 25. The directional mark 45 is set to point towards a degree measurement setting on the trial frame lens gauge (FIG. 5, 112) corresponding to a patient's measured axis of astigmatism.

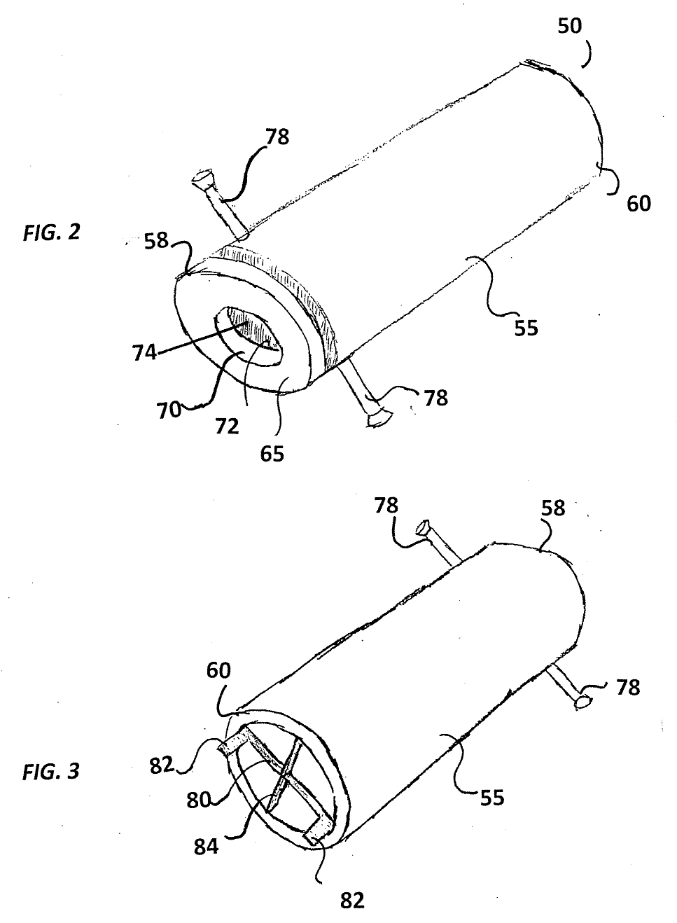

FIG. 2 shows one embodiment of the corneal marking device 50 comprising a housing 55 which, in this example, is preferably a hollow cylindrical tube. The housing 55 has two ends 58, 60. The first end 58 is sealed by a cover 65. The cover 65, in this nonlimiting embodiment, is made of clear plastic, but it can also be made of any such material as long as the material has sufficient transparency. The cover 65 is flat and doughnut-like in shape with a centrally located “hole” or receiving indentation 70. In operation, the receiving indentation 70 receives one end, or tip, of a shaft of the transillumination device (see FIG. 5 125). The receiving indentation 70 is preferably covered with a clear plastic inner layer 72, lying at the base of indentation 74.

In this nonlimiting example, there are two rotational positioning members 78 positioned on opposite sides of the housing 55. In use, the rotational positioning members 78 fit into the linear slots 33 and 35, as shown in FIG. 1, positioned on the guide member 30 of the corneal marking device assembly 20. The rotational position of the corneal marking device 50 within the corneal marking device assembly 20 is fixed according to the marking path relative to the patient's eye.

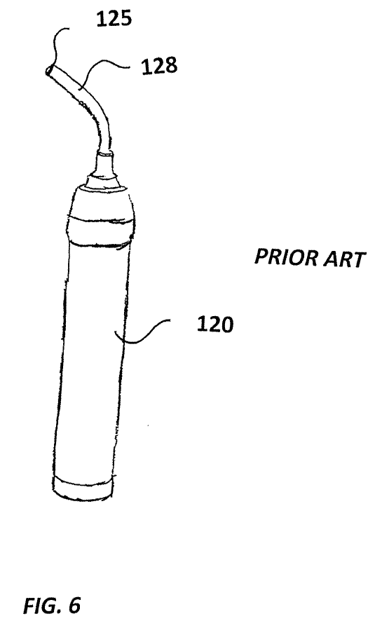

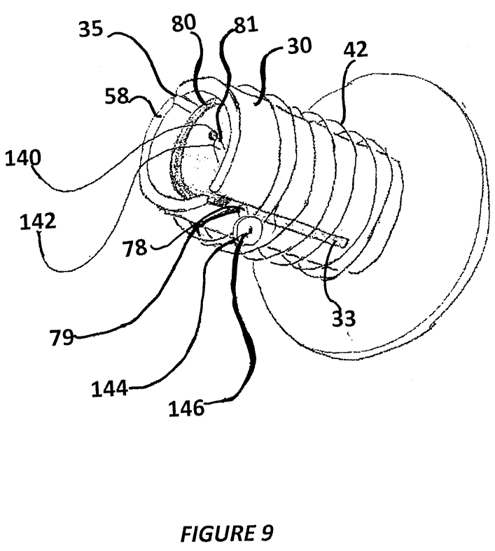

FIG. 3 is a perspective view of the exemplary embodiment of the corneal marking device 50 as shown in FIG. 2 from the opposite end 60. The opposite end 60 has a marking structure 80 radially arranged. The marking structure 80 preferably includes raised edges 82 for co-extensive matching contact with the convex curvature of the outer corneal surface of the eye. Marks on the corneal surface are preferably applied by painting the raised edges 82 of the marking structure 80 with, for example, any pharmacologically acceptable dye, such as gentian violet. Alternatively, the raised edges of marking structure 82 can be sufficiently sharp to make a mark without the need of a dye. A T-intersection with the marking structure 80 forms a crosshair reference 84. The crosshair reference 84 centers the corneal marking device 50 on the corneal light reflex formed by the reflection of the light from the transillumination device FIG. 6, or a LED (FIG. 9 140) onto the cornea.

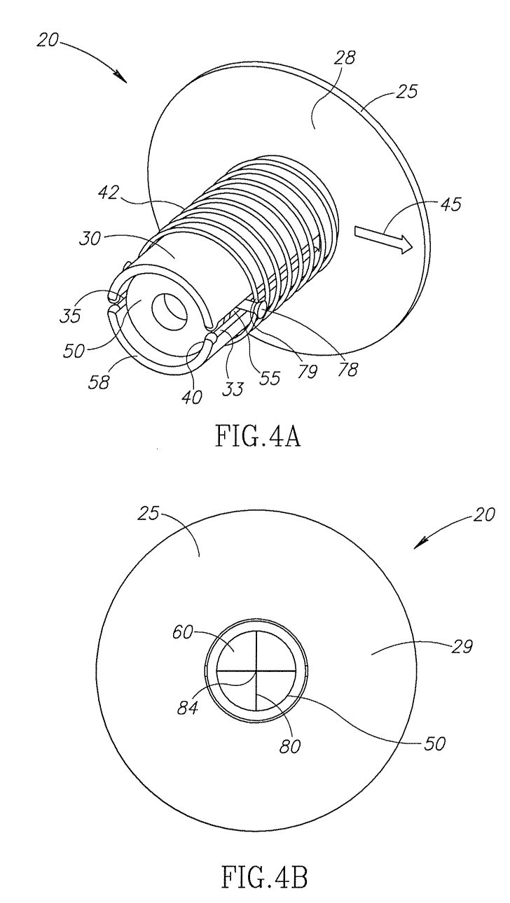

FIG. 4A is a perspective view of one embodiment of the corneal marking device assembly 20 in which the corneal marking device 50 is fitted within the guide member 30. In operation, the corneal marking device assembly 20 is mounted onto the trial frame apparatus 90 (See FIG. 5) with the corneal marking device 50 in situ. In this example, the two rotational positioning members 78 register inside the two linear slots 33, 35 such that the housing 55 of the corneal marking device 50 can freely move in and out of the guide member 30 without rotation relative to the guide member 30. In this nonlimiting example, the two rotational positioning members 78 contact one end of a compression spring 42 such that there is a small resistance to the inward movement of the housing of the corneal marking device 50 inside the guide member 30. The stricture 40 prevents the rotational positioning members 78 and the corneal marking device 50 from coming out of the guide member 30.

FIG. 4B is a view of the corneal marking device assembly 20 as it would appear from the patient's perspective. The circular base 25, as viewed from face 29, is on the opposite side from the perpendicularly arranged guide member 30 on face 28. A hole through the circular base 25 allows passage of the second end 60 of the corneal marking device 50 through the circular base 25 and into close proximity to the eye during the marking method. The marking structure 80 and the crosshairs reference 84 are shown.

FIG. 5 shows an exemplary embodiment of the corneal marking device assembly 20, as shown in FIG. 4A, when mounted to the trial frame apparatus 90. The trial frame apparatus 90 is well known in the art.

In this example, a vertical adjustment control 94 adjusts a vertical position of the trial frame apparatus 90 with respect to the patient's eye. A horizontal adjustment control 97 adjusts a horizontal position of the trial frame apparatus 90 with respect to the patient's eye. Two level adjustment controls 100, 105 adjust the level of a lens holder assembly 110. A rotational adjustment control 115 rotates the corneal marking device assembly 20 with the directional mark 45 pointing to the axis delineated on a trial frame gauge 112. The corneal marking device assembly 20 is held in place by the spring-like action of the lens holder mounting assembly 108 on the trial frame apparatus 90.

The surgeon levels and adjusts the corneal marking device assembly 20 by using the vertical adjustment control 94, horizontal adjustment control 97, level adjustment controls 100, 105, and the rotational adjustment control 115 on the trial frame apparatus, thus setting the marking path for the corneal marking device 50.

FIG. 6 is an exemplary embodiment of a transillumination device 120 as is well known in the art. This nonlimiting example is a means for illuminating the eye to observe a corneal light reflex along the marking path. A fiber optic shaft 128 of the transillumination device 120 has a small bend midway down its length. A tip 125 of the fiber optic shaft 128 directs light into the patient's eye coaxially through the guide member 30 of the corneal marking device assembly 20. As is shown in FIG. 5, in one exemplary embodiment, the tip 125 of the transillumination device 120 is pushed into the receiving indentation 70 at the receiving end 58 of the housing 55. The surgeon, holding the transillumination device 120, applies pressure in the direction of the marking path towards the patient's eye, thus pushing the corneal marking device 50 through the guide member 30 and along the marking path. Alternative means for illuminating the eye to observe a corneal light reflex are contemplated. For example, a wide variety of external light sources may be used as the illumination device in addition to the preferred transillumination device 120. The light source may also be integral with the corneal marking device 50 or corneal marking device assembly 20 or otherwise associated with the mounting apparatus.

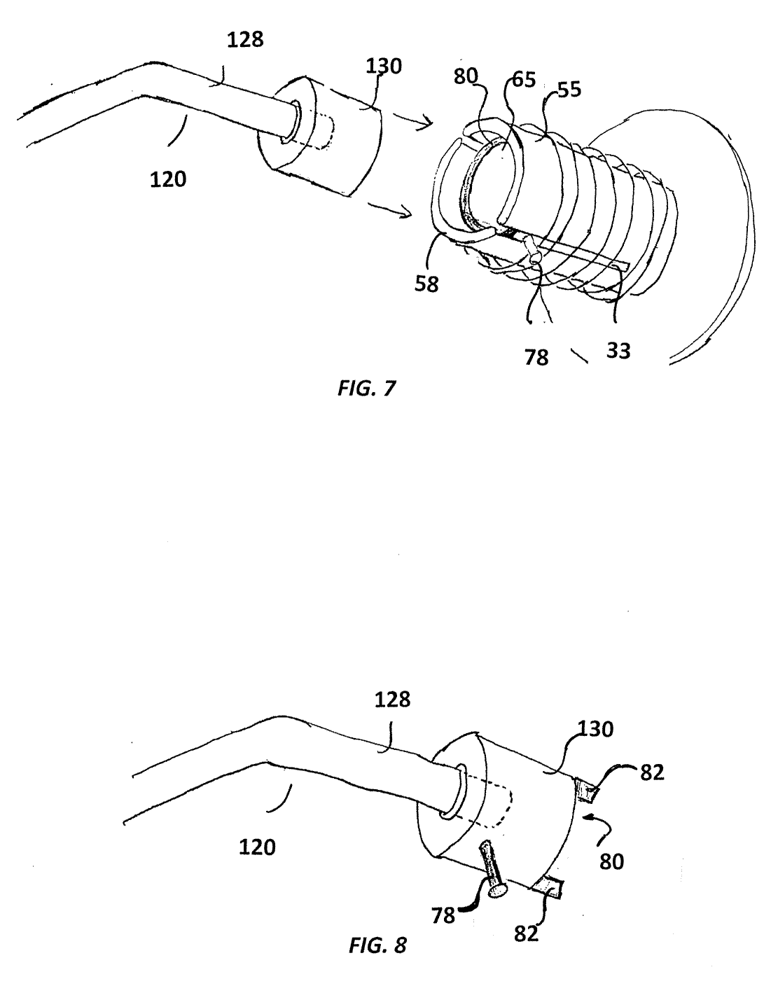

FIG. 7 is an alternative embodiment of the corneal marking device 50. In this example, a bumper 130 is secured to the fiber optic shaft 128 of the transillumination device 120. The receiving end 58 of the housing 55 of the corneal marking device 50 is left open and without a cover 65, as previously described FIG. 2, to receive the bumper 130 of the transillumination device 120. The diameter of the bumper 130 is similar to the diameter of the inside of the housing 55. This is in contrast to the exemplary embodiments shown in FIG. 5 and FIG. 6 in which the tip 125 of the fiber optic shaft 128 is pushed into the covered receiving indentation 70 at the second end 58 of the housing 55. In the present example in FIG. 7 the marking structure 80 and crosshair reference 84 are contained within the guide member 30 of the corneal marking device assembly 20. The bumper 130 engages the marking structure 80 when the transillumination device 120 is pushed by the surgeon into the guide member 30 along the marking path. At least one rotational positioning member 78 kept in register in the linear guide slot 33 fixes the rotational position of the marking structure 80 as it is being pushed through the guide member 30 along the marking path.

FIG. 8 is an alternative embodiment of the corneal marking device 50. In this nonlimiting example the corneal marking device 50 is secured to the tip 125 of the fiber optic shaft 128 such that it can rotate freely on the shaft. The corneal marking device 50 houses the marking structure with 80, 82. At least one rotational positioning member 78 is positioned on the corneal marking device 50. In this embodiment, the guide member 30 of the corneal marking device assembly 20 has no structure relating to the housing 55 of the corneal marking device 50, as was described in the alternative embodiment in FIG. 7. Instead, the corneal marking device 50, positioned to substantially enclose the transillumination device 120, itself housing the marking structure 80. The corneal marking device 50 is pushed by the surgeon into the guide member by the transillumination device 120, sliding through the guide member 30 along a marking path. The rotational alignment of the shaft cover relative to the guide member is fixed by at least one rotational positioning member 78, which can slot into at least one linear slot 33, 35 positioned on the guide member 30.

An alternative embodiment of the corneal marking device assembly 20 and the corneal marking device 50 are shown in FIG. 9. As was previously described above and illustrated in FIG. 4A and FIG. 7, at a first end 79 of the rotational positioning member 78, the rotational positioning member 78 slots into one of a plurality of linear slots 33, 35 on the guide member 30 protruding externally to contact the compression spring 42. In this embodiment however, at least one rotational positioning member 78 at a second end 81 has an internal extension member 142 that extends into the internal space of the guide member 30. A small LED 140 is located on the internal extension member 142 centered within the marking structure 80. A battery compartment 144 and a switch 146, at the first end 79 of the rotational positioning member 78, provide the power switch to illuminate the LED 140. In this embodiment, in contrast to that exemplified in FIG. 4A and FIG. 7, no external light source is necessary. In this example, the means for illuminating the eye to observe a corneal light reflex is provided by the LED 140 fixed within the corneal marking device assembly 20. In operation, the surgeon views the eye through the receiving end 58 of the guide member and through the marking structure 80. The LED is turned to an “ON” position and the patient is instructed to look at the light emitted from the LED 140 as the surgeon adjusts the controls of the trial frame so that the position of the corneal light reflex from the LED is centered within the marking structure 80 (and aligned with the LED 140). The marking structure 80 is advanced by the surgeon by manually pushing the rotational positioning member 78 forward, thus advancing the marking structure 80 inside the guide member 30.

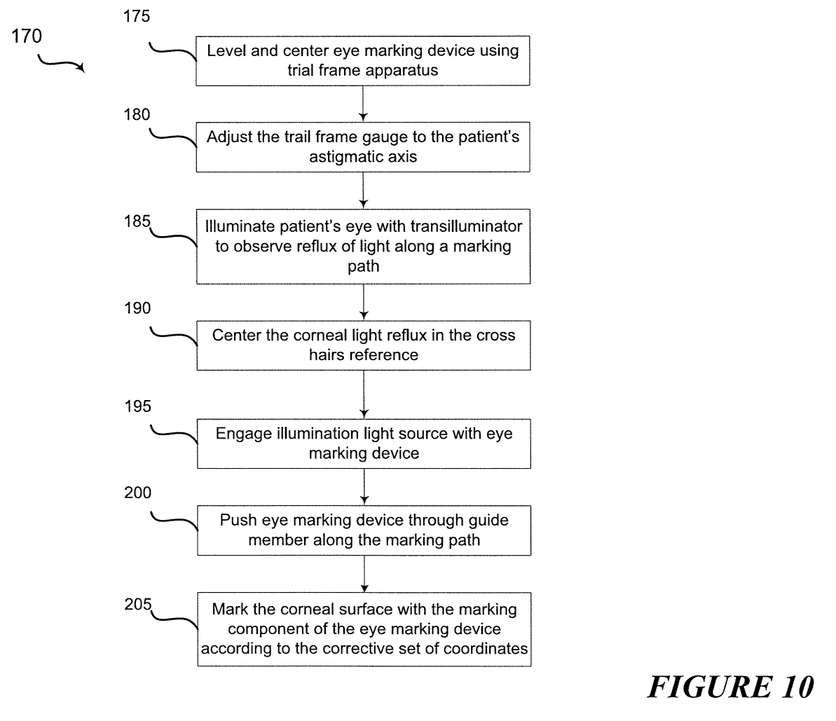

FIG. 10 is a flow diagram of the preferred methodology for corneal marking for corrective eye surgery 170 of the invention as shown in one embodiment in FIG. 5. In this preferred embodiment, the system of the present invention is used with a marking dye, the marking is properly placed on the corneal surface while the patient is preferably sitting at the trial frame apparatus, and the corneal marking device is properly aligned to observe and mark the patient's astigmatic axis. A rough summary of steps are below:

level and center the trial frame and associated marking device using the trial frame position adjustment controls (typically both vertical and horizontal adjustment controls);

adjust the trial frame gauge to the correct astigmatic axis;

illuminate the eye with a light source such as a transillumination device, instructing the patient to look at the light, and observe the resulting corneal light reflect;

adjust the cross hairs (or plurality of centering means) using the trial frame position adjustment controls to center the crosshairs reference on the corneal light reflex;

push the corneal marking device thru the guide while the patient continues to look at the fixation light, thereby providing the corneal light reflect and defining the marking path; and

mark the corneal surface with the corneal marking device.

At a block 175 the trial frame apparatus, as illustrated in FIG. 5, is aligned over the patient's eye so that is centered and level on the patient relative to the corneal marking device housed within the corneal marking device assembly. In this nonlimiting embodiment the means for aligning the eye of a patient in a position relative to the corneal marking device are the controls on the trial frame apparatus 90 itself. As previously described in FIG. 5, a vertical adjustment control 94 adjusts the vertical position of the trial frame apparatus 90 with respect to the patient's eye. A horizontal adjustment control 97 adjusts a horizontal position of the trial frame apparatus 90 with respect to the patient's eye. Two level adjustment controls 100, 105 adjust the level of a lens holder assembly 110.

At a block 180 the rotational adjustment control 115, as shown in FIG. 5, rotates the corneal marking device assembly 20 with the directional mark 45 pointing to the axis delineated on a trial frame gauge 112. The rotational adjustment control 115 orients the corneal marking device based on the astigmatic axis of the patient's eye.

At a block 185, the eye of the patient is illuminated coaxially to the corneal marking device. In a preferred embodiment, the means illuminating the eye to observe the corneal light reflex is a handheld transillumination device 120, as is well known in the art and shown in FIG. 6. Alternative means for illuminating the eye are contemplated. For example, a wide variety of external light sources may be used as the illumination device in addition to the preferred transillumination device 120. The light source may also be integral with the corneal marking device 50 or corneal marking device assembly 20 or otherwise associated with the mounting apparatus. For example, in another embodiment, the light source is an LED 140 positioned within the corneal marking device assembly as illustrated in FIG. 9 and described above. The resulting corneal light reflex can be observed, defining the marking path.

At a block 190, the surgeon controls the trial frame apparatus 94, 97,115 to center and align the crosshairs reference 84, as is shown in one embodiment in FIG. 3, of the corneal marking device 50, with the corneal light reflex.

At a block 195, the corneal marking device 50 is guided along a marking path through the guide member 30 of the corneal marking device assembly 20. The guide member 30 of the corneal marking device assembly, as is shown in a preferred embodiment in FIG. 1, FIG. 4A, and FIG. 5, provides the means for guiding the corneal marking device 50 in a fixed orientation relative to the eye and into contact with the eye along the marking path. In operation, the corneal marking device assembly 20 is mounted onto the trial frame apparatus 90 as is shown in FIG. 5, with the corneal marking device 50 in situ. Referring now to FIG. 5, in this example, two rotational positioning members 78 register inside the two linear slots 33, 35 such that the housing 55 of the corneal marking device 50 can freely move in and out of the guide member 30 without rotation relative to the guide member 30. The rotational positioning member 78 fixes the marking path of the corneal marking device 50, guiding it through the guide member 30 as the surgeon applies pressure with the transillumination device 120. In the alternative embodiment, as described above and illustrated in FIG. 9, at least one rotational positioning member 78 is used by the surgeon to manually advance, or push the marking component 80 through the guide member 30 and into proximity of the patient's eye.

In an alternative embodiment, as is shown in FIG. 5, the tip 125 of the transillumination device 120 engages the corneal marking device 50 by being inserted into a shallow receiving indentation 70 on the receiving end 58 of the corneal marking device 50. With reference to FIG. 5, this exemplary means for engaging the transilluminating light source 120 with the corneal marking device 50, thus engaging the corneal marking device 50, provides the means by which the corneal marking device can be pushed along the marking path or, in other words, the corridor that centers on the corneal light reflected by the light source. In this exemplary embodiment of the corneal marking device 50, the first end 58 is sealed by the cover 65. The cover 65 is shown as flat and doughnut-like in shape, with a centrally located “hole” or receiving indentation 70. The receiving indentation 70 is covered with a clear plastic inner layer 72, lying at the base of indentation 74. In operation, the receiving indentation 70 receives the tip of the shaft of the transillumination device (see FIG. 5, 120). Alternative means for engaging the transillumination device are illustrated in FIG. 7 and FIG. 8, and discussed in detail above. Alternatively, the illumination device may be a LED 140, as described in FIG. 9. In this later example, the LED 140, an alternative light source, is attached to the marking structure 80. The light source is thereby engaged when the surgeon turns “ON” the LED 140 at the switch 146 positioned at one end 79 of the rotational positioning member 78, and manually advances the corneal marking device 50 through the guide member 30 using the rotational positioning member 78.

At a block 200, the corneal surface of the eye is marked by the corneal marking device 50. In the preferred embodiment, the means for marking the eye is provided by the corneal marking device 50 and, specifically, the raised edges 82 of the marking component 84 as shown in the preferred embodiment in FIG. 2, FIG. 3, FIG. 4A and 4B, and FIG. 5, and in alternative embodiments of FIG. 7, FIG. 8, and FIG. 9.

With reference to a preferred embodiment shown in FIG. 3, marking on the corneal surface is achieved by bringing a marking component 80, positioned at second end 60 of the housing 55 into contact with the corneal surface. The marking structure 80 has raised edges 82 for co-extensive matching contact with the convex curvature of the outer corneal surface of the eye. Marks on the corneal surface are preferably applied by painting the raised edges 82 of the marking structure 80 with, for example, any pharmacologically acceptable dye. Alternatively, the raised edges 82 can be sufficiently sharp to make a mark providing the means for marking the eye without the need of a dye. Also shown is the T-intersection, forming with the marking structure 80.

While the preferred embodiment of the invention has been illustrated and described, as noted above, many changes can be made without departing from the spirit and scope of the invention. As described above, the mounting apparatus may be other than a trial frame apparatus, for example, a more general mounting assembly designed for or retrofit to associate with the present invention. Likewise, a wide variety of external light sources may be used as the illumination device in addition to the preferred transillumination device. As described above, the light source may also be integral with the corneal marking device or corneal marking device assembly or otherwise associated with the mounting apparatus. The makes and models of the trial frame apparatus and the transillumination device can vary, the type of materials used to make any of the components can vary, the type of marking dye and marking type can vary, and the particular shape and dimensions of the corneal marking device assembly, bumpers for the transillumination device, and the corneal marking device can all vary according without departing from the spirit and intent of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A corneal marking system having a corneal marking device associated with a mounting apparatus having position adjustment controls, comprising:
a means for illuminating the eye to observe the corneal light reflex produced by the illuminating means;
a means for aligning the corneal marking device with an unobstructed corneal surface according to the corneal light reflex using the mounting apparatus adjustment controls; and
a means for marking the eye.

2. The corneal marking system recited in claim 1, wherein the means for aligning the corneal marking device is a trial frame apparatus.

3. The corneal marking system recited in claim 1, wherein the means for marking the eye is a corneal marking component that receives ink to be delivered to a corneal surface of the eye when in a close proximity with the eye.

4. A corneal marking device assembly for corrective eye surgery, comprising;
a base member configured to mount on a mounting apparatus;
a guide member defining a marking path along which a corneal marking device can pass towards a patient's eye, the guide member having first and second ends, wherein the first end receives the corneal marking device and the second end is attachable to the base member in relative proximity to the patient's eye;
at least one guide slot extending along a length of the guide member, wherein the guide slot orients the corneal marking device in fixed rotational position along a marking path; and
a compression mechanism in physical association with the corneal marking device that provides resistance against the corneal marking device as it moves along the marking path.

5. A corneal device for marking an eye prior to a corrective surgical procedure, comprising:
a housing having two ends the first end having a marking component for marking the corneal surface of the eye and a crosshairs reference to align the corneal marking device and the second end configured to provide a light source defining a marking path associated with the reflex of light reflected by the eye; and
at least one rotational positioning member configured to fix the corneal marking device along the marking path.

6. A method for marking a corneal surface of an eye of a patient, comprising:
orienting a mounting apparatus having position adjustment controls relative to the patient's eye;
illuminating the eye with a light source;
observing the corneal light reflex produced by the light source;
aligning a corneal marking device associated with the mounting apparatus with an unobstructed corneal surface according to the corneal light reflect using the mounting apparatus adjustment controls; and
marking the unobstructed corneal surface of the eye.

7. The method of claim 6 further comprising positioning the patient in a seated position during marking of the corneal surface.
